SEQUENCE LISTING (6 sequences)

SEQ ID NO: 1 - DNA, 213 bp, Homo Sapiens
ggaccggagacgctctgcggggctgagctggtggatgctcttcagttcgtgtgtggagacaggggcttttatttcaacaagcccacagggtatggctccagcagtcggagggcgcctcagacaggtatggtggatgagtgctgcttccggagctgtgatctaaggaggctggagatgtattgcgcacccctcaagcctgccaagtcagcttag

SEQ ID NO: 2 - DNA, 795 bp, Homo Sapiens
ggcgcgagctcggggggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtag

SEQ ID NO: 3 - DNA, 213 bp, Artificial Sequence
  Chemically engineered human IGF-1
ggtcctgagaccctctgcggtgctgagctcgttgatgctctccagttcgtttgcggagataggggtttttacttcaacaagccaaccggatacggttctagcagcagaagggcccctcagactggtatcgtggatgagtgctgcttcaggagctgcgatctcagaaggctcgagatgtactgcgctccactcaagcctgccaagtccgcttga

SEQ ID NO: 4 - DNA, 795 bp, Artificial Sequence
  Chemically engineered human IGFBP-3
ggagctagctctggaggtttgggtcccgtggttaggtgcgagccttgcgatgccagagctctcgcccagtgcgctcctccacccgccgtgtgcgctgagctcgtgagggagcctggatgcggttgctgccttacctgcgctctcagcgagggacagccctgcggtatctacaccgagaggtgcggttccggtcttaggtgccagccttctcccgatgaggccaggcccctccaggctctcctcgatggtagaggactctgcgttaacgctagcgccgttagcaggctcagagcctacctcctcccagctcctccagctccaggaaatgctagcgagtctgaggaagataggagcgccggaagcgtggagagcccctccgtttccagcacccacagggtgtctgatcccaagttccaccccctccactctaagattatcatcatcaagaaaggtcacgctaaggatagccagaggtacaaagttgattacgagtctcagagcactgatacccagaacttctcctccgagtccaagagggagactgaatacggtccctgcaggagagaaatggaggataccctcaatcacctcaagttcctcaatgtgctcagccccaggggtgttcacattcccaactgcgataagaagggattttacaagaaaaagcagtgcaggccttccaaaggtaggaagaggggattctgctggtgcgtggataagtacggtcagcctctcccaggatacaccaccaagggtaaggaggatgttcactgctacagcatgcagagcaagtga

SEQ ID NO: 5 - protein, 70 aa, Homo Sapiens
GPETLCGAELVDALQFVCGDRGFYFNKPTGYGSSSRRAPQTGIVDECCFRSCDLRRLEMYCAPLKPAKSA

SEQ ID NO: 6 - protein, 264 aa, Homo Sapiens
GASSGGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

(19) United States
(12) Patent Application Publication
(10) Pub. No.: US 2008/0199910 A1
(43) Pub. Date: Aug. 21, 2008

(54) Production of recombinant insulin-like growth factor-I (IGF-I) and insulin-like growth factor binding protein-3 (IGFBP-3) in transgenic monocots

(76) Inventors: Samuel Sai Ming Sun, Shatin (HK); Peter Chun Yip Tong, Shatin (HK); Stanley Chun Kai Cheung, Shatin (HK)

Correspondence Address: MORRISON & FOERSTER LLP, 12531 HIGH BLUFF DRIVE, SUITE 100, SAN DIEGO, CA, 92130-2040, US

(21) Appl. No.: 11/677,866

(22) Filed: Feb. 22, 2007

Related U.S. Application Data
(60) Provisional application No. 60/890,828, filed on Feb. 20, 2007.

(51) Int. Cl. C12P 21/00; A01H 5/00; C12N 5/10

(52) U.S. Cl. 435/70.1; 435/419; 800/298; 800/320.2

(57) ABSTRACT

Two important human proteins, insulin growth factor I (IGF-I) and insulin growth factor binding protein 3 (IGFBP-3) have been produced in monocots. The recombinantly produced proteins exhibit the known activities of the native forms.

### CROSS-REFERENCE TO RELATED APPLICATIONS

This application claims benefit of priority to U.S. Provisional Patent Application Ser. No. (“USSN”) 60/890,828, filed Feb. 20, 2007. The aforementioned application is explicitly incorporated herein by reference in its entirety and for all purposes.

### TECHNICAL FIELD

The invention relates to the production of mammalian, especially human, proteins in monocots. This is illustrated by the successful production of IGF-I and IGFBP-3 in transgenic rice.

### BACKGROUND ART

Insulin-like Growth Factor-I (IGF-I) and Insulin-like Growth Factor Binding Protein-3 (IGFBP-3) are important proteins in the regulation of cell and tissue survival, growth and differentiation.

Human IGF-I is a single polypeptide chain of 70 amino acid residues, encoded by a single gene on chromosome 12. It has 48% amino acid sequence identity with proinsulin and insulin. IGF-I contains three intrachain disulfide bridges, at A20-B18, A6-A11 and A7-B6, but no glycosylation site. The majority of circulating IGF-I is synthesized in the liver, and is regulated by growth hormone (GH), insulin and nutritional intake. Circulating IGF-I levels are relatively stable, mainly due to its constitutive pattern of secretion and the binding of IGF-I to high-affinity binding proteins. However, abnormalities in the regulation of IGF-I have been suggested to play a role in the development of insulin resistance and other metabolic abnormalities.

The precise role of IGF-I in the regulation of carbohydrate metabolism has yet to be defined, but it is clearly important in regulating metabolism. In healthy volunteers, recombinant human IGF-I (rhIGF-I) can exert an acute hypoglycemic effect, though rhIGF-I was 10 times less potent than equivalent dose of insulin. In contrast, IGF-I has a more pronounced effect than insulin on protein metabolism, decreasing overall net amino acid flux compared to equivalent glucose lowering doses of insulin. Furthermore, IGF-I is a potent inhibitor of pancreatic insulin release. In patients with type 1 diabetes, the use of rhIGF-I results in improved circulating IGF-I level, reversal of GH hypersecretion, reduction in insulin requirement and improvement in glycemic control. Higher doses of rhIGF-I were required in patients with type 2 diabetes to reduce fasting plasma glucose, insulin and C-peptide levels. In addition, rhIGF-I treatment was associated with a reduction in fat mass, which may partly explain the above-noted beneficial effect on insulin sensitivity.

Six human IGF-binding proteins have been identified. Of the six binding proteins, IGFBP-3 binds more than 95 percent of the IGF-I in serum. It contains 264 amino acid residues, with a calculated molecular weight of around 29 kD. There are three potential N-glycosylation sites (Asn-X-Ser/Thr) located at Asn⁸⁹, Asn¹⁰⁹ and Asn¹⁷² in the IGFBP-3 central region, but carbohydrate units appear not to be essential to IGF binding. The IGF-I/IGFBP-3 dimer forms a ternary complex with the “acid-labile subunit,” which prolongs the half-life of IGF-I and titrates the supply of IGF-I to its receptors.

IGFBP-3 is a 40 to 45 kDa glycoprotein produced locally in many tissues, where it acts as an autocrine and paracrine regulator in modulating cellular growth and apoptosis. IGFBP-3 inhibits cell proliferation and survival by binding to IGF's and prevents them from activating IGF-I receptors on target cells. IGFBP-3 has also been found to regulate cell proliferation negatively and induces apoptosis in an IGF-independent manner. In the absence of IGF-I, IGFBP-3 is able to interact with a number of growth-inhibitory proteins and agents, such as p53, retinoic acid, tumor necrosis factor-α and transforming growth factor-β. Overexpression of IGFBP-3 inhibits cell proliferation and reduces tumor formation with only minor inhibition on the growth of normal organs. Recent studies illustrated that IGFBP-3 inhibits breast cancer, prostate cancer, lung cancer, ovarian cancer and colorectal cancer, so IGFBP-3 is effective as an anticancer agent.

Thus, both human IGF-I and human IGFBP-3 have established pharmaceutical value. A major obstacle to the use of both IGF-I and IGFBP-3 is their cost of production. These proteins are mainly produced recombinantly from microbes such as Escherichia coli; or are extracted from sarcoma cell lines or erythroid cells and/or produced in transgenic mice. These systems are not appropriate for large scale production because of high equipment and production costs and the potential contamination with pathogens. IGF-I and IGFBP-3 occur in other mammals as well, and serve similar functions.

Plant cells can be engineered to accept and express genetic information from a wide range of organisms, including genes from prokaryotic and eukaryotic sources. Since plant cells are eukaryotic, they are able to produce mammalian proteins with the appropriate post-translational modifications (e.g., glycosylation, prenylation and formation of disulfide bridges) often necessary for proper protein or enzyme function. In addition, the seeds of many plant species are edible and it is possible to accumulate the recombinant proteins in seeds. In some instances, the recombinant proteins may not require further processing and purification prior to oral delivery, provided dosage level and frequency are controlled, since each protein has characteristic acid and protease resistance. Delivery vehicles such as bioencapsulation and plant tissues may be used to prevent degradation of protein in the stomach and gut (Daniell, H., et al., Trends Plant Sci. (2001) 6:219-226). Seed-based platforms have also been developed for the biological assembly and production of pharmaceutical proteins (Sardana, R. K., et al., Transgenic Res. (2002) 11:521-531). Their results suggest that the use of plant seeds as a vehicle to produce and deliver biopharmaceuticals via the ‘seed as pill’ route is a viable option.

Recombinant human IGF-I and recombinant human IGFBP-3 have been produced in transgenic tobacco and these results have been reported in three poster presentations at the 5th Hong Kong Diabetes and Cardiovascular Risk Factors, East Meets West Symposium, October 2003 (“Expression of Human Insulin-like Growth Factor I (IGF-I) and Insulin-like Growth Factor Binding Protein 3 (IGFBP-3) in Transgenic Tobacco”); the 64th Scientific Sessions of American Diabetes Association, June 2004 (“Plants as Bioreactors for Expressing Human Insulin-like Growth Factor I (IGF-I) and Insulin-like Growth Factor Binding Protein-3 (IGFBP-3)”); and the 2004 annual meeting of the American Society of Plant Biologists, July 2004 (“Transgenic Expression of Human Insulin-like Growth Factor Binding Protein-3 (IGFBP-3) in Tobacco Seeds”). The production of these proteins in the tobacco plant does not offer the advantages of production in such edible monocots as corn, rice, wheat and barley. Monocots also include such important food crops as sugar cane, pineapples, dates and bananas. These monocot plants represent fertile sources of edible materials.

Rice, consumed daily by over 40% of world's population, is well-established in agricultural practices worldwide, and is recognized as model bioreactor to produce pharmaceutically and commercially important proteins and vaccines (Fischer, R., et al., Transgenic Res. (2000) 9:279-299). It does not contain noxious chemicals such as nicotine and toxic alkaloids as does tobacco, and has low allergenicity. Recombinant protein in rice can accumulate up to 1% grain weight. Using specific promoters and signal sequences to target production and storage of recombinant protein in the endosperm region of the rice (which amounts up to 91% of the total seed grain), protein accumulation can be increased to 2.7% of the grain weight (Liu, Q. Q., Thesis Department of Biology (2002) Yangzhou University, China and the Chinese University of Hong Kong, Hong Kong). A single rice plant can have up to 100 tillers producing over 10,000 grains which allows rapid production of large amounts of seeds and recombinant proteins. In addition, fast growing (3-4 round per year) japonica subspecies of rice can be used as bioreactor plants for production. Storage and distribution of the dried seeds are simple, over 5 months of storage at room temperature does not show significant loss of yield and activities of recombinant proteins in the grains (Stoger, E., et al., Plant Mol. Biol. (2000)42:583-590). In conditions of low moisture contents the grains can be stored from three to five years without losing viability (Huang, N., BioProcess International (2002) January :54-59).

Production in alternative grains, such as oats, may be more appropriate if the proteins are designed for use in livestock.

Previous studies have shown that codon usage biases are strongly correlated with gene expression levels. Highly expressed genes preferentially use a subset of codons called “optimal” codons (Moriyama, E. N., et al., J. Mol Evol (1997) 45:514-523). Moreover, these optimal codons correspond to the most abundant tRNA's, leading to enhanced translation accuracy and efficiency (Marais, G., et al., J. Mol Evol (2001) 52:275-280). DNA sequences of the human IGF-I and IGFBP-3 were modified in the Examples below based on the plant preferred codons used in two seed storage proteins, in an attempt to enhance the protein production of these proteins in rice. Codon usage of lysine-rich protein (LRP) from winged bean and methionine-rich 2S albumin (PN2S) from Paradise nut were chosen as the bases for modification because high expression and stable accumulation (3-10% and 3-15% of total extractable seed proteins for LRP and PN2S, respectively) in transgenic Arabidopsis was observed in previous studies.

Another strategy to increase yield of the target protein used in the Examples below is to direct the recombinant protein to the specific compartments in order to prevent degradation by the proteolytic system of the cells. It has been reported that attachment of signal peptide sequences leading to the endoplasmic reticulum (ER) secretary pathway and the tetrapeptide KDEL, which is the ER retention signal at the N- and C-terminal ends of a foreign gene are generally required for high levels of accumulation of its product (Wandelt, C. I., et al., Plant J. (1992) 2:181-192; and Herman, E. M., et al., Planta (1990) 182:305-312). The KDEL tetrapeptide contributes to protein localization by interaction with a receptor that recycles between the Golgi complex and the ER. The KDEL signal is sufficient for soluble protein accumulation in the plant ER (Wandelt, C. I., et al., supra; Frigerio, L., et al., Plant Cell (2001) 13:1109-1126; and Napier, J., et al., Planta (1997) 203:488-494). It has been found that scFv levels were 6-14 times higher in cells transformed with the construct containing KDEL than without KDEL (Conrad, U., et al., Plant Mol. Biol. (1998) 38:101-109).

### DISCLOSURE OF THE INVENTION

The invention provides an economic and practical source of two important mammalian proteins—IGF-I and IGFBP-3. Production of the human forms is preferred. By producing these proteins in monocots, the present invention provides, for the first time, a useful source able to provide sufficient quantities in a form adaptable to human therapeutic use, or to use in veterinary contexts.

Thus, in one aspect, the invention is directed to monocotyledonous cells that have been modified to produce human IGF-I and/or human IGFBP-3. In another aspect, the invention is directed to plants or plant parts comprising such cells.

### BRIEF DESCRIPTION OF THE DRAWINGS

FIG. 1 shows the nucleotide sequence encoding human IGF-I as reported in Jansen, M., et al., Nature (1983) 306:609-611.

FIG. 2 shows the nucleotide sequence encoding human IGFBP-3 as reported by Wood, W. I., etal., Mol. Endocrinol. (1988) 2:1176-1185.

FIG. 3 shows the nucleotide sequence encoding human IGF-I as modified for codon preference in plants.

FIG. 4 shows the nucleotide sequence of human IGFBP-3 modified according to codon preference in plants.

FIG. 5 shows the amino acid sequence of human IGF-I.

FIG. 6 shows the amino acid sequence of human IGFBP-3.

FIG. 7 shows the results of an assay showing the ability of human IGF-I produced in rice to effect ruffling in L6 cells, and the susceptibility of this effect to commercial human IGFBP-3.

FIG. 8 shows the effectiveness of human IGFBP-3 produced in rice to inhibit the growth of MCF-7 cells.

### MODES OF CARRYING OUT THE INVENTION

As illustrated below, for the first time, two important human proteins, IGF-I and IGFBP-3 have been produced in monocots. As the monocots include major sources of nutrition and are free of noxious compounds, they are ideal for the production of proteins intended for human treatment. They are also ideal for production of corresponding proteins in treating herbivorous or omnivorous mammals. Production of these proteins can be enhanced by appropriate design of DNA constructs comprising expression systems having the nucleotide sequences encoding these proteins operably linked to appropriate control sequences for their expression.

Techniques for effecting genetic modification of plant cells and reconstituting intact plants have been well known in the art for some time. See, e.g., Gelvin, et al., Plant Molecular Biology Manual, (1990)); Dashek, Methods in Plant Biochemistry and Molecular Biology (CRC Press 1997). A useful summary of the state of the art in this respect is found in U.S. Patent Publication 2004/0009476, published 14 Jan. 2004, incorporated herein by reference with respect to its disclosure of appropriate techniques for genetic manipulation of plants.

Once transformed plant cells containing the recombinant constructs are obtained, transgenic plants can then be regenerated therefrom, and evaluated for level of desired protein production.

Several techniques can be used to optimize expression of non-native nucleotide sequences in plants. As further described in the examples below, the encoding nucleotide sequences can be modified according to codon preferences for expression in plant cells. Such modification is based on published data describing codon preferences in plants. Second, the encoding nucleotide sequences may be extended to add signal and retention sequences and direct the encoded protein to the endoplasmic reticulum and effect its retention. This, too, has a favorable effect on yield. In general, signaling peptides are generated at the N-terminus of the desired protein and retention signal at the C-terminus thereof.

Using these techniques, the yields of the desired IGF-I and IGFBP-3 are appreciably enhanced.

It is further useful to employ appropriate promoters to effect expression. For instance, suitable promoters include 35S CaMV, rice actin promoter, ubiquitin promoter, or nopaline synthase (NOS) promoter. Tissue-specific promoters to enhance production in seeds includes the seed-specific glutelin promoter (Gt-1ₚᵣₒ) but other seed-specific promoters may also be used. Termination signals may also be employed, such as the nopaline synthase termination signal.

Two groups of constructs were designed as shown below to drive the plant-optimized encoding sequences and introduced into rice by Agrobacterium-mediated transformation. One group of constructs contains the glutelin signal peptide (SP) alone while the other contains SP together with the targeting tetrapeptide KDEL signal. These constructs were synthesized to increase the expression of protein and to target the proteins into compartments for storage as well as to increase protein stability. Seed-specific glutelin promoter (Gt-1ₚᵣₒ) was used to drive the expression of IGF-I and IGFBP-3 in transgenic rice, and the expression of the transgenes was analyzed. The produced rhIGF-I and rhIGFBP-3 from transgenic rice grains were biologically active.

The constructs may also be modified to include aids in purification, such as histidine tags or FLAG sequences and/or to include markers such as fluorescent proteins so that purification can be followed. Cleavage sites may be engineered between the encoded protein and purification tag and/or marker as well. Standard purification techniques may be employed if desired, or in some instances, plant tissues may be administered orally to take advantage of the nutritive value of the plant and its low toxicity.

The recombinantly produced IGF-I is used to reduce insulin requirements and improve glycemic control in subjects with either type 1 or type 2 diabetes. IGFBP-3 has been shown to effect apoptosis and is useful in the treatment of malignancies. Thus, if desired, the recombinantly produced proteins may be formulated into compositions for administering to subjects in need of treatment with these proteins. General methods for formulating proteins and other pharmaceuticals may be found in Remington's Pharmaceutical Sciences, latest edition, Mack Publishing Co., Easton, Pa., incorporated herein by reference. The proteins are typically administered systematically either parenterally by injection or transdermal or transmucosal delivery or may be, in some cases, administered orally. Various formulations designed for particular modes of administration may be used, including liposomal formulations, and formulations containing nanoparticles based on lipids or polymers and the like.

The following examples are offered to illustrate but not to limit the invention.

### EXAMPLE 1

### Transformation of Agrobacteria with IGF-I and IGFBP-3 Constructs

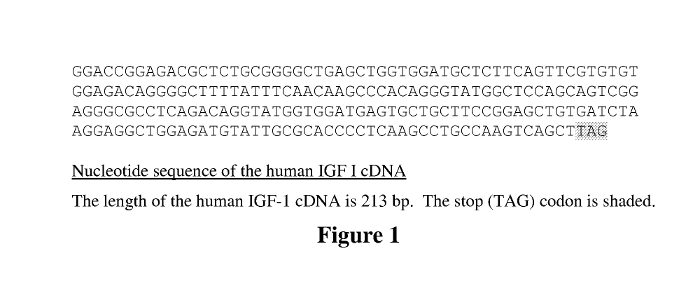

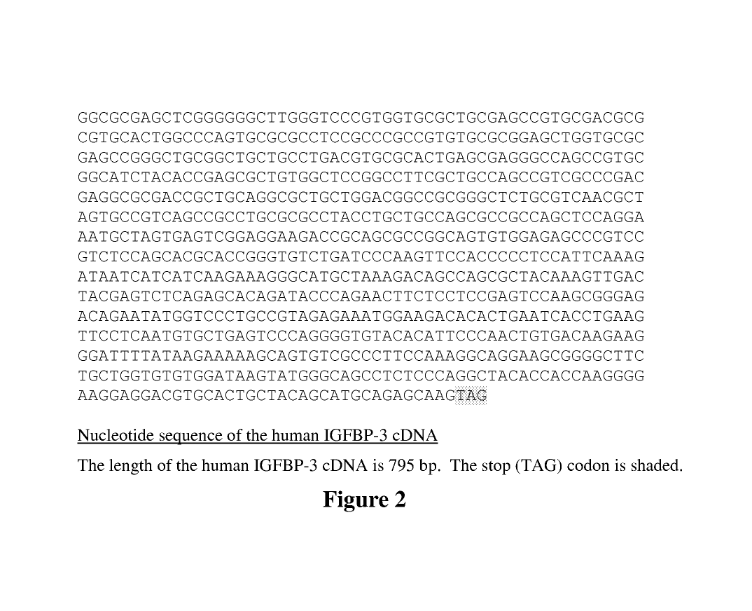

The DNA sequences of the human IGF-I (FIG. 1) and IGFBP-3 (FIG. 2) were modified based on the preferred codons used in two seed storage proteins. Codon usage of lysine-rich protein (LRP) from winged bean and methionine-rich 2S albumin (PN2S) from Paradise nut (Table 1) were chosen as the bases for modification because high expression and stable accumulation (3-10% and 3-15% of total extractable seed proteins for LRP and PN2S, respectively) in transgenic Arabidopsis was observed in previous studies.

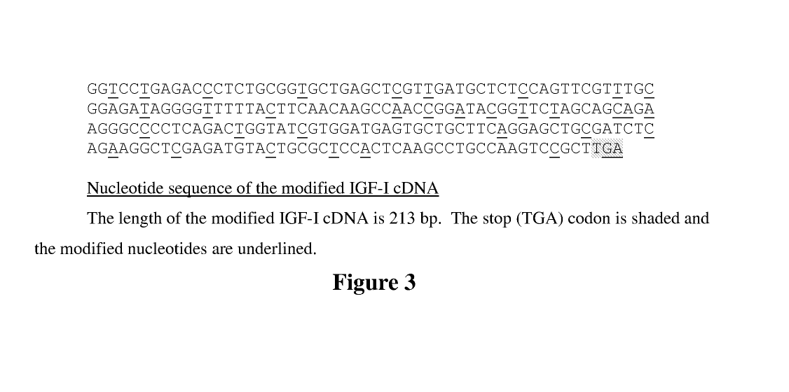

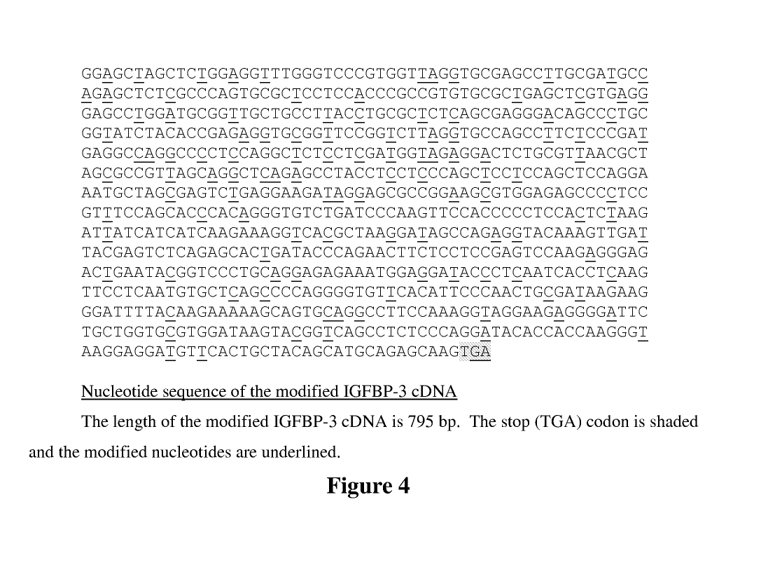

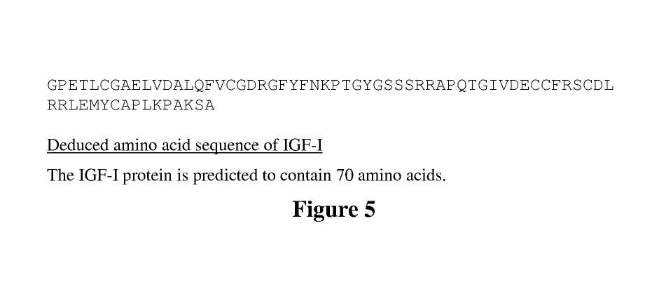

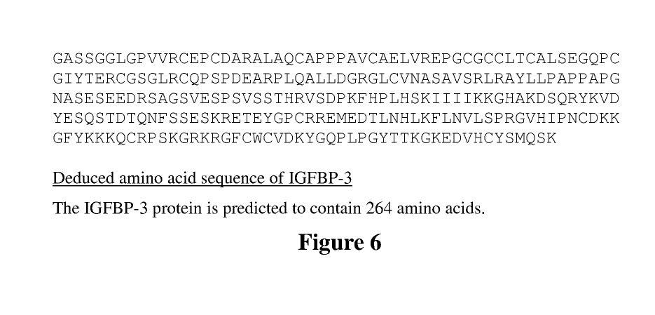

The modified IGF-I (FIG. 3) and IGFBP-3 (FIG. 4) genes, encoding the same amino acid sequences (FIGS. 5 and 6) as the original ones, were obtained from MWG Biotechnology Company. The change of codons of IGF-I and IGFBP-3 were 28.6% and 14.8% respectively.

TABLE 1
Summary of codon preference prioritized based on the
LRP and PN2S sequences

| | Triplet | | |
|---|---|---|---|
| Amino acid | 1^{st} priority | 2^{nd} priority | 3^{rd} priority |
| Phe | TTT/TTC | — | — |
| Leu | CTC | TTG | CTT/CTG |
| Ile | ATT/ATC | ATA | — |
| Met | ATG | — | — |
| Val | GTT | GTG | — |
| Ser | AGC | AGT/TCT/TCC | TCA |
| Pro | CCT/CCC | CCA | CCG |
| Thr | ACC | ACA | ACT |
| Ala | GCT | GCC | — |
| Tyr | TAC | — | — |
| His | CAC | — | — |
| Gln | CAA | CAG | — |
| Asn | AAT/AAC | — | — |
| Lys | AAA/AAG | — | — |
| Asp | GAT | GAC | — |
| Glu | GAG | GAA | — |
| Cys | TGC | — | — |
| Trp | TGG | — | — |
| Arg | CGG/AGA/AGG | CGC | — |
| Gly | GGC/GGA | GGT | GGG |
| *** | TGA | — | — |

Constructs were designed to enhance rhIGF-I and rhIGFBP-3 stability and yield, and to control their glycosylation. Two protein targeting signals were added to direct the target proteins to specific compartments in rice grain, either glutelin signal peptide (SP) for glycosylation in the Golgi apparatus or the tetrapeptide KDEL for stable accumulation without glycosylation in the endoplasmic reticulum. The expression constructs were driven by seed-specific glutelin promoter (Gt-1ₚᵣₒ). The details of the chimeric gene constructs are as follows,

IGF-1:

(Gt-1ₚᵣₒ)+SP+IGF-I*+NOSₜₑᵣ 1)

(Gt-1ₚᵣₒ)+SP+IGF-I*+KDEL+NOSₜₑᵣ 2)

IGFBF-3:

(Gt-1ₚᵣₒ)+SP+IGFBP-3*+NOSₜₑᵣ 3)

(Gt-1ₚᵣₒ)+SP+IGFBP-3*+KDEL+NOSₜₑᵣ 4)

wherein * indicates modified cDNA of IGF-I or IGFBP-3, and NOSₜₑᵣ indicates the nopaline synthase terminator.

The chimeric gene cassettes were inserted into the super-binary vector pSB130, and transformed into Agrobacterium strain, EHA 105, which is ideal for infection of rice callus. A simple freeze-thaw method was used to transform the Agrobacterium (as described in Chen, H., et al., BioTechniques (1994) 16:664-668, 670). Transformed bacteria were selected with antibiotics hygromycin (50 mg/L), and DNA transformation of the target genes was further confirmed by PCR.

### EXAMPLE 2

### Transformation, Selection and Cultivation of Rice

Scutellum from mature seeds of japonica 9983 were excised and cultured on callus inducing medium (N₆ basal medium, 2 mg/l 2,4-D, 0.5 g/l casein hydrolysis, 30 g/l sucrose, 2.5 g/l Phytagel®, pH 5.8) for callus induction. After 4-7 days of culturing in callus induction medium, the calli derived from the immature embryos were immediately used for co-culture with Agrobacterium EHA 105 containing the target genes.

The Agrobacterium was first inoculated in 3 ml LB broth supplemented with 50 mg/L rifampicin and 50 mg/L kanamycin at 28° C. overnight, and then subcultured in 25 ml AB medium (3 g/l K₂HPO₄, 1 g/l NaH₂PO₄, 1 g/l NH₄Cl, 0.3 g/l MgSO₄.7H₂O, 0.15 g/l KCl, 10 mg/l CaCl₂.2H₂O, 2.5 mg/l FeSO₄.7H₂O, 5 g/l glucose, pH 7.2) for 5 hours, followed by centrifugation and resuspension in 15-25 ml AAM medium (AA basal medium, 68.5 g/l sucrose, 36 g/l glucose, 0.5 g/l casein hydrolysis, pH 5.2, 100 μmol/l acetosyringone).

The rice calli were immersed in the Agrobacterium culture for 10-20 minutes at room temperature, with discontinuous shaking. The infected calli were then transferred into a N₆D₂C medium (N₆D₂, 10 g/l glucose, pH 5.2) containing 100 μmol/l acetosyringone for 3 days at 26-28° C. in the dark. After co-culture, the calli were put onto N₆D₂S medium (N₆D₂, 50 mg/l hygromycin B, 500 mg/l cefotaxime, pH 5.8) for selection of resistant calli at 26° C. in the dark for 2 weeks, followed by culture in new N₆D₂S medium until the newly-formed resistant calli came out.

The resistant calli were then put onto the HGPR medium (Higrow® Rice Medium (GIBCO-BRL), 50 mg/l hygromycin B, 200 mg/l cefotaxime) for 7 days in the dark and 7 days in the light at 26° C. After pre-regeneration, the resistant calli were transferred to the MSR medium (MS basal medium, 30 g/l sucrose, 0.5 g/l casein hydrolysis, 2 mg/l 6-BA, 0.5 mg/l NAA, 0.5 mg/l KT, pH 5.8, 50 mg/l hygromycin B, 500 mg/l cefotaxime, 2.5g/l Phytagel®) for regeneration at 26° C. for 16 h in light/8 h in the dark.

When the regenerated shoots came out, they were put onto the ½ MSH medium (½ MS macro salt, Fe-EDTA and micro salts, MS vitamins, 30 g/l sucrose, 0.5 mg/l NAA, pH 5.8, 50 mg/l hygromycin B, 500 mg/l cefotaxime, 2.5 g/l Phytagel®) for rooting. Transgenic rice plantlets were finally transferred to soil and grown in the greenhouse.

### EXAMPLE 3

### Analysis of Transgenic Rice

Southern Blot Analysis

The transgenic rice plants were analyzed to confirm the integration of target genes into the rice genome. Leaf genomic DNA was extracted by the cetyltrimethylammonium bromide (CTAB) method (Doyle, J. D., et al., Focus (1990) 12:13-15). Fifteen pg of genomic DNA was digested overnight with BamHI, separated on 0.8% agarose gel and transferred to positively charged nylon membrane (Roche) using the VacuGeneXL Vacuum blotting System (Pharmacia Biotech). Hybridization and detection were carried out according to the method described in the DIG Nucleic Acid Detection Kit (Roche). Double strand DIG-labeled DNA probes (IGF-I and IGFBP-3) were prepared using DIG DNA labeling Kit (Roche) by PCR. The probes were heated to denature at 99° C. before use.

The results confirmed the presence of the constructs in the rice genome.

Western Blot Analysis

Total seed protein was extracted from mature rice seeds by grinding the seeds into powder and mixing with protein extraction buffer (50 mM Tris-HCl pH 6.8, 0.1 M NaCl and 10% SDS). After centrifugation, the clear supernatants were transferred to a new Eppendorf tube and saved as seed total protein extract. Different amounts of total protein were then resolved in 17% Tricine SDS-PAGE and blotted on PVDF membrane. Western blot analysis was performed using anti-human IGF-I or IGFBP-3 polyclonal antibodies. Finally the blot was subjected to non-radioactive detection with chemiluminescent Starlight™ Substrate (ICN) as described in the manual of Aurora™ Western Blot Chemiluminescent Detection System (ICN).

The results confirmed the presence of both IGF-I and IGFBP-3 protein in the seed extracts.

### EXAMPLE 4

### Biological Activity of rhIGF-I Produced in Rice

Similar to insulin, IGF-I has been shown to cause membrane ruffle and glucose uptake in the muscle cells. IGFBP-3 can bind to IGF-I to form an ALS complex, which will inhibit the membrane-ruffling effect caused by IGF-I. Recombinant hIGF-I produced in rice was tested in parallel with insulin to compare their biological activities.

Rat L6 skeletal muscle cells expressing c-myc epitope-tagged glucose transporter 4 (GLUT4) (L6myc cells) were maintained in myoblast monolayer culture in α-minimal essential medium containing 10% (v/v) fetal bovine serum (FBS) and 1% (v/v) antibiotic-antimycotic solution (100 U/ml penicillin G, 10 mg/ml streptomycin and 25 mg/ml amphotericin B) in an atmosphere of 5% CO₂ at 37° C. Cells were subcultured by trypsinization of subconfluent cultures using 0.25% trypsin. For differentiation into myotubes, myoblasts were plated in medium containing 2% (v/v) FBS at approximately 4×10⁴ cells/ml to allow spontaneous fusion. Medium was changed every 48 hours and myotubes were used 5-7 days after plating. Rat L6 muscle cells were then grown to the stage of myotubes on 25-mm-diameter glass coverslips placed in six-well plates.

Myotubes were deprived of serum for 3 hours and treated with different concentrations and combinations of rhIGF-I and rhIGFBP-3 extracted from transgenic rice for 10 minutes at 37° C. After these incubations, myotubes were fixed with 3% (v/v) ice-cold paraformaldehyde in PBS for 20 minutes, then washed with 0.1 M glycine in PBS for 10 minutes, permeabilized with 0.1% (v/v) Triton X-100 in PBS for 3 minutes and then washed with PBS. The myotubes were blocked in 0.1% BSA for an hour, followed by incubating in phalloidin (1:500 in 0.1% (w/v) BSA). Following this incubation the cells were washed with PBS and mounted in ProLong antifade solution onto glass slides. Samples were examined with a Zeiss Axioplan 2 imaging microscope and a Zeiss LSM 510 META laser scanning confocal microscope (Carl Zeiss, Jena, Germany).

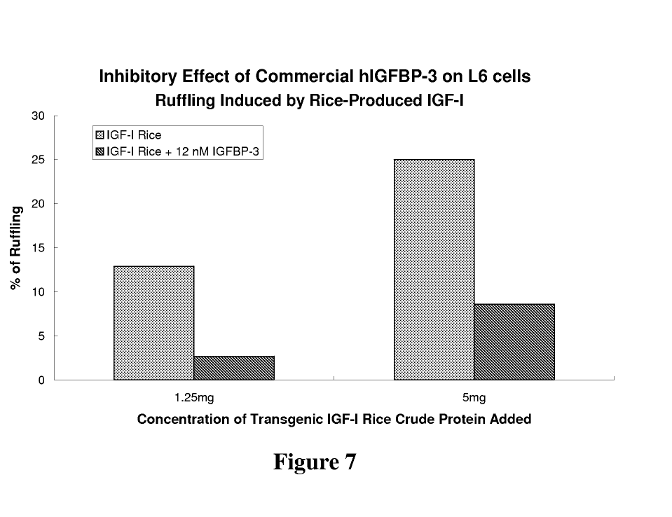

The crude protein of transgenic IGF-I rice causes membrane ruffle of L6 cells and the ruffling effect could be greatly reduced by commercial hIGFBP-3. These results are shown in FIG. 7. As shown in FIG. 7, IGF-1 produced in rice effects ruffling in a dose-dependent manner at 1.25 mg and 5 mg. This activity was inhibited by 12 nM concentration of commercially obtained IGFBP-3.

### EXAMPLE 5

### Anti-Cancer Activity of rhIGFBP-3 Protein Produced in Rice

Human IGFBP-3 has been shown to inhibit the proliferation of oestrogen-dependent and -independent breast cancer cells. In order to examine whether the rhIGFBP-3 produced in rice has the anti-cancer function, MCF-7 human breast cancer cells were employed.

MCF-7 human breast cancer cells were routinely maintained in Eagle's minimum essential medium, supplemented with 1% penicillin and streptomycin, 0.1% fungizome together with 5% fetal bovine serum (FBS) at 37° C. with a humidified atmosphere of 5% CO₂. For rhIGFBP-3 treatment, MCF-7 cells were seeded in 96-well plate with serum-containing medium. Different concentrations of crude protein extracted from rice transformant containing rhIGFBP-3 were added to the cells after 1 day. The crude protein extracts were replaced with fresh ones every 3 days for two times.

After 7 days treatment, the cells were collected for MTT assay. Briefly, 20 μl MTT (3-(4,5-dimethylthiazol-2-yl)-2,5-diphenyl tetrazolium bromide) solution (5 mg/ml MTT in HPBS, pH 7.4) was added to each well and further incubated for 2 hours at 37° C. One hundred μl acid isopropanol was added to each well to break down the cells and to dissolve the purple crystals. The intensity of purple color in each well was measured by using a microplate spectrophotometer at OD₅₇₀.

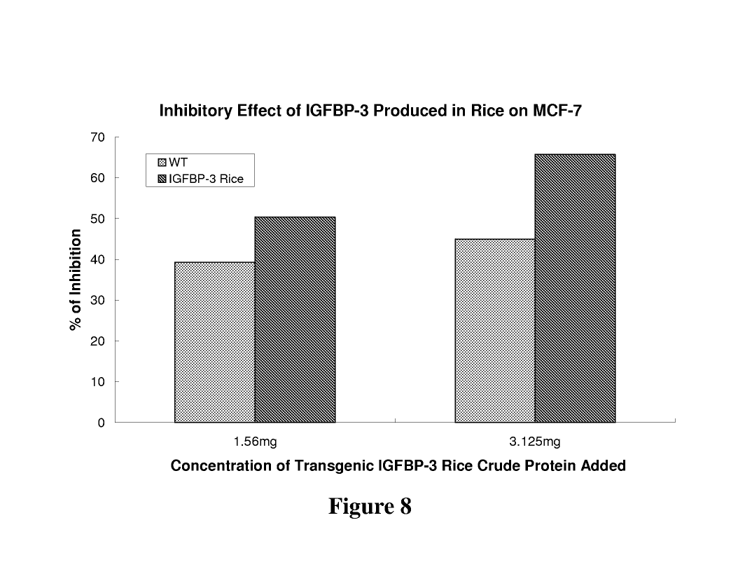

As shown in FIG. 8, the IGFBP-3 produced in rice was able to enhance the inhibition of growth of MCF-7 cells at concentrations of 1.56 mg and 3.125 mg in a dose-dependent manner.

### EXAMPLE 6

### Purification of Recombinantly Produced Proteins

The crude extracts described in the previous examples are further purified using affinity chromatography. Each of IGF-I and IGFBP-3 are applied to chromatographic columns which columns are coupled to their respective antibodies for further purification. Impurities are washed through the column, and proteins eluted by adjusting the pH and salt concentration.

Purification is made simpler by modifying the constructs of Example 1 to include a marker protein.

### EXAMPLE 7

### Enhancement of Recombinant Protein Yield

Seeds are obtained from the plants described in Example 2 and replanted for second and third generation crops. Enhanced yields of the recombinant IGF-I and IGFBP-3 are obtained.

1. Monocot plant cells which have been modified to contain a DNA construct for expression of a nucleotide sequence encoding insulin-like growth factor I (IGF-I) or a nucleotide sequence encoding insulin-like growth factor binding protein-3 (IGFBP-3), wherein said DNA constructs comprise said encoding nucleotide sequences operably linked to control sequences for their expression in monocot plant cells.

2. The cells of claim 1, wherein said IGF-I and IGFBP-3 are human proteins.

3. The cells of claim 1 or 2, wherein said control sequences comprise a promoter operable in seeds of monocots and termination sequences operable in seeds of monocots.

4. The cells of claim 1 or 2, wherein said encoding nucleotide sequences are extended at the N-terminus by nucleotide sequences encoding a signal peptide leading to the endoplasmic reticulum (ER) secretory pathway.

5. The cells of claim 1 or 2, wherein said encoding nucleotide sequences are extended at the C-terminus ends by nucleotide sequence encoding an ER retention signal.

6. The cells of claim 1 or 2, which are rice cells.

7. A transgenic plant or plant part which comprises the cells of claim 1 or 2.

8. Monocot plant cells that comprise IGF-I or IGFBP-3.

9. The cells of claim 8, wherein the IGF-I and IGFBP-3 are human.

10. The cells of claim 8 or 9, which are rice cells.

11. A plant or plant part that comprises the cells of claim 8 or 9.

12. The plant part of claim 11, which is seed tissue.

13. The plant or plant part of claim 11 which is rice.

14. The plant part of claim 13, which is seed tissue.

15. A method to produce IGF-I or IGFBP-3 in monocots which comprises culturing the plant cells of claim 1 or 2.

16. A method to produce IGF-I or IGFBP-3 in monocots which comprises culturing the plant of claim 11.

17. The method of claim 16 wherein the plant is rice.
